(19) United States
(12) Patent Application Publication
(10) Pub. No.: US 2025/0195892 A1
(43) Pub. Date: Jun. 19, 2025

(54) METHOD FOR EARLY ILLNESS DETECTION USING IMPLANTED NEUROSTIMULATION SYSTEM FEATURES

(71) Applicant: BIOTRONIK SE & Co. KG, Berlin (DE)

(72) Inventors: Pamela Shamsie Victoria Riahi, Portland, OR (US); Andrew B. Kibler, Lake Oswego, OR (US); Sean Slee, Tigard, OR (US)

(21) Appl. No.: 18/849,716

(22) PCT Filed: Mar. 28, 2024
(86) PCT No.: PCT/EP2023/057992

§ 371 Date: Sep. 23, 2024

Related U.S. Application Data
(60) Provisional application No. 63/324,259, filed on Mar. 28, 2022.

(30) Foreign Application Priority Data
Apr. 25, 2022 (EP) 22169735.2

(51) Int. Cl. A61N 1/36; A61N 1/02; A61N 1/372; G16H 50/30

(52) U.S. Cl. CPC A61N 1/36139; A61N 1/025; A61N 1/37258; G16H 50/30

(57) ABSTRACT

An implantable neurostimulation device includes a system for determining at least one physiological and/or at least one biological parameter. The device is configured to derive a health status based at least in part on the at least one parameter. The system includes at least one element or shares at least one element associated with a neurostimulation therapy for determining the at least one parameter. Further aspects relate to a method carried out by such a device and a computer program.

The present invention generally relates to an implantable neurostimulation device and a method and a computer program for operating such a device, particularly for deriving a health status of a patient by the device.

Neurostimulation devices that can be implanted into a patient for facilitating therapies and treatment of various diseases have been known. For example, their application may involve the treatment of neurologic disorders, pain conditions, movement disorders, epilepsy.

Common implantable neurostimulation devices may comprise one or more leads with an electrode system which may be coupled to the patient's nervous system. The electrode system may be connected to a main unit, which may comprise a power supply, as well as a control unit to control the electrical stimulation delivered by the electrode system to the nervous system. The electrical stimulation may be configured to purposefully modulate a nervous activity for treatment of a particular medical condition.

In known devices the electrical stimulation may be set up as an open-loop stimulation, wherein the patient may manually adjust the stimulation characteristics based on his subjective perception, e.g. a pain perception. Implantable neurostimulation devices may also be configured for closed-loop stimulation, wherein the patient's body position sensed by the device may be used to automatically adjust neurostimulation characteristics based thereon.

However, known neurostimulation devices do not fully harness the potentials enabled by such devices being implanted into patients.

Therefore, there is a need to improve neurostimulation devices and their use.

The aspects described herein address the above need at least in part.

A first aspect of the present invention relates to an implantable neurostimulation device. The device may comprise a system for determining at least one physiological and/or at least one biological parameter. The device may be configured to derive a health status based at least in part on the at least one parameter. The system (S) comprises at least one element or shares at least one element associated with a neurostimulation therapy for determining the at least one parameter.

An underlying idea is that patients having a neurostimulation implant for the treatment of a particular medical disease may be significantly impacted by various other illnesses. Because of their potentially weakened health which requires them to undergo neurostimulation therapy they may be considered a particular vulnerable group of patients. As an example, these patients may have an impaired sense of determining their health due to their affected nervous system such that feelings of sickness and symptoms do not appear as severe as they are. In addition, the neurostimulation itself may interfere with the subjective health perception of the patient.

The above aspect harnesses the neurostimulation device and its prominent placement within the patient's body which may access various physiological and/or biological parameters, to enable an overall assessment of the patient's health status in addition to the neurostimulation capabilities of the device. The at least one parameter may be associated with a physiological and/or biological parameter of the patient having the implant. Based thereon, the patient and his health status may be extensively monitored regarding various health aspects depending on the chosen at least one parameter for deriving the health status. Moreover, the health status of the patient may be derived based on quantitatively determined data and not merely on a subjective symptom assessment by the patient. To that regard, the inventive concept highly benefits the already medically vulnerable group of patients requiring neurostimulation implants. These patients may be in particular need to track their health status quantitatively since their subjective perception of symptoms may be impeded by their weakened health and/or due their neurostimulation therapy which provides them with electrical modulation of the nervous system. The above aspect enables this patient group to objectively determine their health status which may highly benefit their current and long-term health, e.g. due to reduced mental stress, early prevention possibilities.

As a further advantage, the patient may not require an additional implant to track his/her health. Hence, the inventive concept may greatly minimize the need for unnecessary surgery which avoids putting the patient under additional high medical stress. With the inventive device, the patient may be provided with a neurostimulation device, as well as an internal health tracker by only one surgical procedure.

The health assessment of patients may be of particular importance as various outbreaks of known or new diseases are still possible worldwide and their impact may not be underestimated. For example, a pandemic of a new virus (e.g. Covid-19) may require the need for enhanced diagnostic capabilities for optimized care of infected patients. In this regard, an early detection of sickness may be optimal to the health outcome of the patients, especially for patients with a pre-existing condition associated with a weak immune system (e.g. patients with diabetes, cardiovascular diseases and/or other chronic conditions).

A viral infection may affect the nervous system in such ways that the feelings of sickness and symptoms do not appear as severe as they actually are, such as in case of subclinical hypoxia where patients do not feel the usual shortness of breath associated with lack of oxygen circulation even though they are experiencing it. This may lead to subjective minimization of (the severity of) their health condition, late disease detection and more severe short and long-term organ damages, or even death, for example.

In particular, the population of patients with neurostimulation devices may be of high risk to that regard since they are generally older and typically present one or more chronic conditions (e.g. diabetes-related neuropathy). These conditions put them at higher risk of severe health impact of any virus infection (e.g. SARS-CoV-2 virus). It is therefore especially important for their health outcome to be able to detect a sickness early. Viral testing (e.g. by nasal swabs, PCR tests, etc.) may only be performed manually by medical personnel or the patient which usually happens when serious symptoms have already appeared or worsened. Sick patients may not get tested early because they minimize their symptoms, believe they will recover without issues and/or they want to avoid medical consequences. Overall, these circumstances may lead to a late diagnosis of the viral disease. Although early sickness detection is extremely relevant for viral infections (e.g. SARS-CoV-2) the same rationale applies for any sickness in general in the neurostimulator-implanted population.

The inventive concept addresses the above specifics, by providing a neurostimulation device with a system for determining at least one physiological and/or biological parameter (e.g. temperature, electrocardiogram, etc.) that can be predictors of upcoming physical symptoms such as fever or may correlate with the health status of the patient to derive his/her health status.

Implementing a system for determining at least one physiological and/or at least one biological parameter, wherein the system comprises at least one element or shares at least one element associated with a neurostimulation therapy provides the advantageous effect of being able to derive a health status using an implantable neurostimulation device having reduced technical complexity, leading typically to a reduction of space, computing power and increased device reliability. For instance, a temperature sensor which is integrated into the an implantable pulse generator can of a neurostimulator can be used for sensing the body temperature of a patient at different times of the day. The temperature data can be used to derive a general health state of the patient, either by a processing unit of the neurostimulator, or by transmitting the temperature data to an external computational device, where the data is evaluated. In that example, the temperature sensor can e.g. share the same processing unit and/or the same battery with other units of the neurostimulator which are associated with neurostimulation therapy, as for instance the stimulation unit or the telemetry unit.

Compared to a sensor which would not share a unit with the neurostimulator which is associated with neurostimulation therapy, the proposed solution is less power consuming, because energy or computational resources of the sensor are shared with the neurostimulator. If a computational unit is shared, the data of the sensor would be directly available at the neurostimulator, without necessity of additional communication pathways or additional devices.

As an example, the system for determining may be configured for sensing at least one physiological and/or biological signal of the patient having the implant. The at least one signal may be sensed over a predetermined time (e.g. 1 second, 1 hour, 1 day or 1 week, etc.) which may be a time associated with determining a corresponding physiological and/or biological parameter. For example, the signal A may be sensed over a time/period of 10 seconds which may be required at a minimum to determine a corresponding parameter A. The corresponding parameter may be determined by the system from the sensed signal by applying signal processing to the sensed signal. The signal processing may be carried out by hardware (e.g. by a microcontroller, microprocessor, ASIC etc.) and/or software comprised in the system and/or the device. There is also possibility that a part of the signal processing/analysis takes place outside of the device (i.e. remotely), e.g. on a server part of a remote service center (in the cloud).

In another embodiment, a predetermined time is not needed, but just a minimal period of time to allow for data processing. Instead, data is continuously collected, processed and used to update the predictions of an artificial intelligence methods (e.g. machine learning model).

The health status or predicted/future status (e.g. prediction of future health status changes with artificial intelligence methods using physiological measures) may be derived based on a characteristic of the at least one parameter, wherein the at least one parameter may be medically associated with the corresponding health status. In one example the health status may be derived based on merely one physiological and/or biological parameter. For example, the health status may be derived based on a determined parameter A. In another example the health status may be derived based on a combination of a plurality of physiological and/or biological parameters. For example, the health status may be derived based on a set of determined parameters A, B, or A, B and C. This concept may enable a modular approach for patient specific health assessment. For example, for a particular patient group only certain parameters may be deemed medically relevant. Hence, this approach may ensure the reduction of complexity, computing power and/or avoiding unnecessary hardware configurations.

The system for determining the at least one physiological and/or biological parameter may comprise elements or share elements associated with the neurostimulation therapy (e.g. hardware, software, data input/output, etc.) for determining the at least one parameter. For example, it may use an element (e.g. microprocessor) required for neurostimulation therapy for a different function, namely determining the at least one parameter for deriving the health status. In other examples, the system for determining may be a separate (physical or logical) entity which is not necessarily configured to adapt the neurostimulation therapy. In this case the system may have its own elements and/or configuration (e.g. hardware, software, data input/output, etc.) which are not required for the neurostimulation therapy. However, it is understood that also in this case, the system may be essentially comprised by a housing of the neurostimulation device.

The derived health status may comprise one or more medical conditions which correspond to the health of the patient. The device may be configured to derive said medical condition(s) based at least in part on the at least one physiological and/or biological parameter. As an example, the medical condition may be a disease, an illness, an unhealthy condition, or a significant deviation from the norm of one or more physiological and/or biological parameters. Further the medical condition may be a current or future medical condition (e.g. machine learning algorithms predicting future illness onset based on physiological measures). To illustrate an example, the derived health status may comprise a medical condition MC1 (e.g. an illness 1, based on parameter A), a medical condition MC2 (e.g. a significant deviation of parameter A), a medical condition MC3 (e.g. a significant deviation of parameters B, C and D) and/or a medical condition MC4 (e.g. an unhealthy condition, based on parameters B and C).

The health status may comprise various categories or groupings of the medical conditions to provide a structured overview of the health status e.g. for an easy readout. The categories or groupings may be based on types of diseases, illnesses or the physiological and/or biological parameter they are based on. For example, group X may comprise medical conditions associated with the heart, group Y may comprise medical conditions associated with parameter B.

It is noted that the implantable neurostimulation device is understood herein as any implantable device that may have an interface with the nervous system and/or may provide a neurostimulation function, for example a spinal cord stimulator (SCS), a cochlear implant, a visual implant, etc.

In an example, the health status may comprise a likelihood of a medical condition. Notably, the device may be configured to derive the likelihood and/or an onset of a medical condition based at least in part on the at least one parameter. Specifically, it is considered that it may not be possible for all cases to derive an exact medical condition (or health status) based on the determined at least one parameter by the system. This may only be possible via an extensive medical analysis which may require a doctor, sample analysis, external medical equipment, etc. The benefit of the invention is that it may provide a quantitative tracking function of the parameter activity inside the patient which may correlate with a likelihood and/or an onset of a medical condition. A suspicious parameter activity and/or a significant deviation from the norm may point to an early stage of the medical condition. The device may be configured to implement an algorithm to detect when a criterion for a (high) likelihood of the medical condition is met.

There is also the possibility of parameters being used with artificial intelligence methods (e.g. machine learning models) to predict high likelihood of medical condition existence, onset or future onset (the important point being that the parameter may not display medical condition onset right away, but shows parameter behavior that machine learning models interpret as predicting a future onset).

In an example, the device may be configured to derive the health status based at least in part on a signature and/or a statistic of the at least one parameter over a predetermined period of time and/or at least one of a value, set of values, pattern or computed property. The signature may be defined or automatically learned (e.g. with artificial intelligence methods). Notably, the device may be configured to derive the medical condition and/or the likelihood/onset (or future onset) of the medical condition based at least in part on the signature and/or the statistic of the at least one parameter over a predetermined period of time.

The signature may be a specific pattern, a characteristic curve, a shape, a (significant) change, a shift, a computed property, etc. of the at least one parameter or a combination of parameters. The signature may also comprise a correlation of multiple parameters with each other.

The statistic may comprise one or more statistics related to the at least one parameter over the predetermined period of time. For example, the statistic may be a mean value, an average value, a standard deviation, a frequency content, an intensity etc. of the at least one parameter (e.g. a signal power or an integral) or a variety of parameters.

The predetermined period of time, a predetermined minimum period of time and/or a parameter-specific minimum period of time may be specific to the at least one parameter and/or the medical condition and/or the health status (which the neurostimulation device intends to derive from the at least one parameter). In another example, the predetermined period of time may be specific to the signature and/or the statistic required to derive the health status. For example, the signature SA of parameter A (e.g. a characteristic pattern of parameter A) may be determined over a predetermined period of time t_{A} (e.g. 1 hour), whereas the statistic SB of parameter B (e.g. a mean value of parameter B) may be determined over a predetermined period of time t_{B} (e.g. 1 day).

In an example, the system may be configured to determine the at least one parameter based at least in part on a predetermined timing. For example, the at least one parameter may be determined regularly, e.g. in predetermined time intervals. The inventive concept considers that it may be effective to determine the at least one parameter not permanently but in separate readouts, e.g. every hour, every week, every month, every year, etc. This may significantly reduce system complexity and/or reduce consumed power by the device. In accordance, the health status may be derived for each readout of the at least one parameter based on the predetermined timing, for example. The predetermined timing may be parameter specific and/or specific to the associated health status and/or medical condition. For example, health status A may require the characteristics of parameters A, B and C, wherein it may be medically relevant to determine the health status (and thereby the associated parameters A, B and C) once every hour.

In another example parameters are not collected regularly but are instead sent at irregular intervals based on device/hardware/patient constraints, and/or are processed outside the device (e.g. in a remote service center) and/or used to update instantaneously (as it receives new data) an artificial intelligence method (e.g. machine learning model/algorithm) that predicts the health status/future status.

It is also possible that a parameter may be determined based on a set of parameters which were determined based at least in part on the predetermined timing. For example, the parameter A may be determined regularly once every hour over a day, which accumulates to a set of 24 parameters (or parameter values) after one day. Said set of 24 parameters (or parameter values) may be used to derive an associated parameter B and/or may be used to derive the health status of the patient.

In an example, the system may be configured to determine an abrupt and/or a gradual change of the at least one parameter. Additionally or alternatively, this change and/or the at least one parameter may be fed into a machine learning algorithm that predicts health status changes (current or future). Notably, the abrupt and/or gradual change may be understood herein as a change in medically relevant time frames which may relate to the at least one parameter, and/or the one or more associated medical conditions and/or health status (e.g. illnesses, sicknesses). For example, the classification of an abrupt change of parameter A may be satisfied, if a significant change (e.g. by more than a predetermined threshold) occurs over 1 day. In another example, an abrupt change of parameter B may be satisfied if the significant change occurs over 1 hour. The abrupt and/or gradual change may also be based on an assessment of the derivative of the at least one parameter over a time.

In an example, the device may be configured to derive the health status based at least in part on comparing the at least one parameter with a parameter specific threshold. The inventive concept thus comprises the evaluation of a threshold condition being met by the at least one parameter. For example, the health status may be based on the evaluation that the at least one parameter is above, below, or equal to a parameter specific threshold. Depending on the resulting threshold relation (e.g. parameter A being above parameter specific threshold TH_{A}, i.e. A>TH_{A}) a respective health status may be derived. In another example, the threshold condition may be based in addition on a predetermined time to derive the health status. For example, the at least one parameter may have to be above (or below or equal to) the parameter specific threshold for a certain time period (e.g. 6 hours). In another example the evaluation of the threshold condition may only take place after a certain time associated with a particular event has passed (e.g. 1 hour after the neurostimulation device was charged, 1 second after physical activity of the patient is detected, etc.). Hence, the effects of (extrinsic) events altering the properties of the at least one parameter are greatly minimized which may significantly reduce false positives.

In another example, the health status may be derived based on a plurality of evaluations of a set of threshold conditions. For example, the set of threshold conditions may be evaluated in an evaluation window comprising multiple separate evaluations of the set of threshold conditions. In that respect the health status may be based on a certain proportion of thresholds being crossed over said evaluation window. For example, in the first evaluation step a set of M parameters may be compared to their respective thresholds with a determination of the amount of respective threshold crossings (e.g. M=10, wherein 2 thresholds were crossed in the first step, meaning 20% threshold crossings). The evaluation step may be repeated multiple times EW (e.g. EW=24), ideally in equally spaced apart time intervals (e.g. 1 hour), to span the evaluation window (e.g. 1 day). If over the course of the evaluation window a certain proportion of thresholds were crossed a corresponding health status may be determined. The proportion of threshold crossings may be evaluated in total over the evaluation window. For example, with M=10, and EW=24, a total of 240 threshold conditions may have been evaluated during the entire evaluation window. If in this example, the proportion of threshold crossings for deriving the corresponding health status would be 50%, the corresponding health status would be based on 120 threshold crossings over the entire evaluation window. In another example, deriving the health status may be dependent on a number of evaluation steps which individually exceed a proportion of threshold crossings. For example, the critical proportion of threshold crossings may be 50% for one evaluation step. The critical number of evaluation steps which are allowed to have 50% threshold crossings respectively may be six. In this case, if seven evaluation steps in the evaluation window individually have at least 50% threshold crossings, a corresponding health status may be derived.

In an example, the device may be configured to monitor the health status. The device may thus be configured to derive the health status in an automatic manner continuously or periodically (e.g. every second, every hour, etc.). This may avoid manual input from the patient and/or doctor to determine the health status. Further, the device may be configured to store the derived health status (e.g. comprising various medical conditions) over a prolonged period of time. The monitoring which may be combined with the storing of the respective health status may thus enable the creation of a health status history of the patient.

Additionally or alternatively, the/a system may receive measured parameters from the/a device, since deriving a health status and collecting parameter data may happen on different media (e.g. physiological data sent from a device to a remote service center where complex processing and/or health status predictions are made.

In another example, the device may be configured to monitor the health status, and a system that received measured parameters from the device may thus be configured to derive the health status (e.g. in an automatic manner continuously or periodically), since health status and parameter data collection can happen on different media (e.g. physiological data sent from a device to a remote service center where complex processing/health status predictions are made).

Further, the device may be configured to monitor the health status to collect parameter values. In one example, the monitoring function may not take place in the device, but the device will always at least collect some data.

The device may be further configured to wirelessly communicate with an external device, a device at a surface of the body of the patient, and/or a further implant to enable a sending of the medical status and/or the at least one parameter to the (external) device. This may enable an easy readout of the health status by a doctor without surgical intervention. In an example, the health status may be regularly sent to the (external) device. The communication may be via intra-body communication, WiFi, NFC, Bluetooth, etc.

In an example, the device may be configured to trigger an alert based at least in part on the health status. The alert (which may comprise the health status data) may be sent to the (external) device, e.g. for the notification of the patient and/or medical personnel (e.g. via relaying the alert, by the (external) device to the medical personal). The medical personal may subsequently assess/decide if the patient should come into a health care facility for check-up, testing, and/or diagnosis depending on the alert/health status. The alert may also be stored on the device itself for a later readout, for example if temporarily the notification cannot be sent to the external device.

In an example, the system may comprise at least one sensor. The sensor may be used to detect a physiological and/or a biological signal of the patient. It may be coupled to the patient and the neurostimulation device in various ways. For example, the sensor may be connected from its sensing position (e.g. a certain body area, a body organ, etc.) to the device over a wired connection. In another example the sensor may be wirelessly coupled to the neurostimulation device. The sensor may also be enclosed in the neurostimulation device, e.g. by a housing of the latter. The sensor and/or elements of the sensor may also be on the outer surface of the neurostimulation device itself (e.g. on the surface of the main unit, the housing, etc.). In some examples, the system may comprise one or more sensors that are connected to the implantable (implanted) neurostimulation device over a wired connection, enclosed in the implantable (implanted neurostimulation device, and/or arranged on the outer surface of the implantable (implanted) neurostimulation device.

In an example, the sensor may comprise at least one of the following: a temperature sensor, an evoked compound action potential sensor, an impedance sensor (e.g. a tissue impedance sensor), a cardiac sensor, an activity sensor, an accelerometer.

Activity may be measured with an accelerometer, and accelerometers can be used to measure a variety of other metrics besides activity such as body position, gait pattern, step count. The activity sensor may potentially be relying on something different than accelerometry, such as pedometers.

The temperature sensor may be configured for sensing the body temperature of the patient and/or sensing the temperature of a specific body part/organ of the patient. The evoked compound action potential (eCAP) sensor may be for sensing an electrical response of a group of neurons to an electrical stimulation (e.g. a stimulus by an electrode system of the neurostimulation device). In an example, the eCAP sensor may be configured to sense the spinal cord neural activity. The tissue impedance sensor may be configured to sense the electrical impedance of a tissue inside the patient. It may be based at least in part on one or more neurostimulation leads comprising the electrode system of the neurostimulation device. For example, the impedance may be measured between pairs of electrodes on at least one neurostimulation lead, between electrodes of different neurostimulation leads and/or between at least one electrode and the main unit (and/or an implanted pulse generator) of the neurostimulation device. The cardiac sensor may be a far-field electrical sensor for sensing heart activity. The cardiac sensor and/or the system for determining may be configured to determine the heart rate, as well as the heart rate variability. The activity sensor could be implemented by or comprise an inertial sensor, an accelerometer and/or a piezo sensor detecting body vibrations and/or a rate of rotation, for example.

In an example, the at least one physiological and/or biological parameter may comprise at least one of the following: a body temperature, an evoked compound action potential amplitude, an evoked compound action potential duration, an evoked compound action potential frequency component, a tissue impedance, a heart rate, a heart rate variability, a body movement intensity, a daily step count, a gait, a body position, a sleeping pattern, a sleeping quality.

The range of properties of evoked compound action potential (eCAPs) may be used as parameters. There might be future more complex signal properties that could potentially be used as an input, such as the average power of the signal, the integral, etc. There can be for example eCAP signal measured (e.g. amplitude, frequency, duration) and computed/processed (e.g. integral, power, etc.) properties.

The gait may, for example, comprise a walking bout, a walking bout frequency, a walking speed, walking harmonics. The body position may, for example, comprise various positions, for example sitting, laying down (e.g. supine, prone, side), standing, walking, driving. The at least one parameter may further comprise a time spent in the respective body position, as well as an acceleration signal corresponding to the respective body position. There can also be activity metrics, e.g. accelerometry-based activity counts, to assess general activity/body movement. The sleeping pattern may comprise various sleeping pattern parameters, for example a time, a sleeping period, a sleeping body position and/or a sleeping body movement (e.g. turning, tossing, body position adjustment, rotations). It may further comprise an occurrence frequency of a sleeping pattern parameter, a duration of time between sleeping movements, a sleeping movement intensity and/or a sleeping movement orientation, and/or other computer sleep quality metrics that combines one or more of the parameters listed above.

In an example, the at least one physiological and/or biological parameter may comprise a perception threshold, and the system may be configured to determine the perception threshold based at least in part on a manual input to the system. Additionally or alternatively, the system may be developed in which the perception threshold is automatically estimated using eCAPs. For example, the perception threshold (PT) may be understood as a measure of a minimum stimulation input (e.g. a lowest stimulation amplitude) by the neurostimulation device necessary to generate a perceptible sensation for the patient. The minimum stimulation input may be associated with at least one set of stimulation settings.

The perception threshold may be regularly determined during standard clinical practices, wherein the result may be manually inputted to the system of the neurostimulation device by the patient and/or medical staff. For example, changes in perception thresholds may indicate changes in the health status of the patient.

A second aspect relates to a method carried out by an implantable neurostimulation device. The method may comprise determining at least one physiological and/or at least one biological parameter. The physiological and/or biological parameter is determined using a system, wherein the system comprises at least one element or shares at least one element associated with a neurostimulation therapy for determining the at least one parameter; It may further comprise deriving a health status based at least in part on the at least one parameter. The raw data and/or above mentioned parameter may be collected by the implant, but processing/predictions/analyses may be carried out in the device or remotely (service center, cloud).

A third aspect relates to a computer program comprising instructions to perform the method when the instructions are executed by a computer. In an example, the computer program instructions may be stored on a non-transitory computer-readable storage medium. For example, the computer program may be stored on a storage medium of a neurostimulation device (e.g. a spinal cord stimulator, etc.), as described herein. The computer program may allow an autarkic, automated implementation of the aspects described herein. Consequently, technical intervention from medical staff and the patient may be minimized.

Additionally or alternatively, the program can actually be stored outside a device, in a remote service center or cloud connected directly or indirectly to the device. The device sends raw or pre-processed data to the remote service center or cloud where the program operates directly, or where the program from another platform extracts the data to operate.

A fourth aspect relates to a use of a neurostimulation device implanted into a human for deriving a health status of the human. The neurostimulator device comprises a system for determining at least one physiological and/or at least one biological parameter. The system comprises at least one element or shares at least one element associated with a neurostimulation therapy for determining the at least one parameter. The health status is derived based at least in part on the at least one parameter.

It is noted that the method steps as described herein may include all aspects described herein, even if not expressly described as method steps but rather with reference to an apparatus (or device). Moreover, the devices as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor, all possibly located in a cloud.

Using the methods and embodiment described herein, a list of health outcomes to monitor may include, but are not limited to:
Pocket/Generalized infection
Sepsis
Pain progression/new pain
Arthritis progression
Cardiac arrythmias (e.g. bradycardia, tachycardia, atrial fibrillation, conduction block)
Sudden fall
Lack of exercise/sedentary lifestyle
Viral or bacterial infections

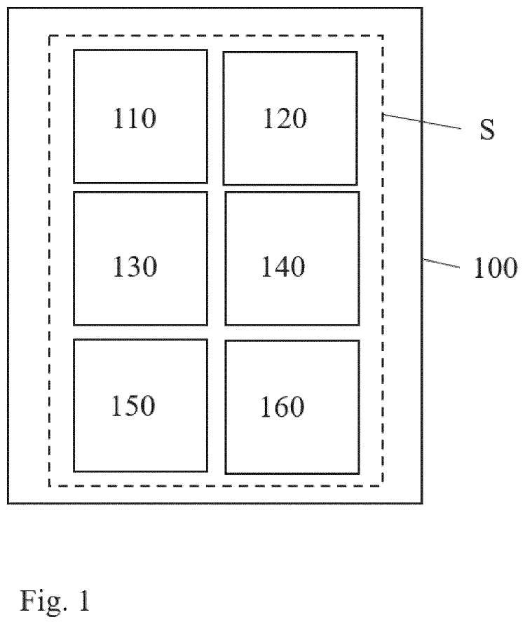

FIG. 1 Schematic representation of an exemplary embodiment of a neurostimulation device according to the present invention.

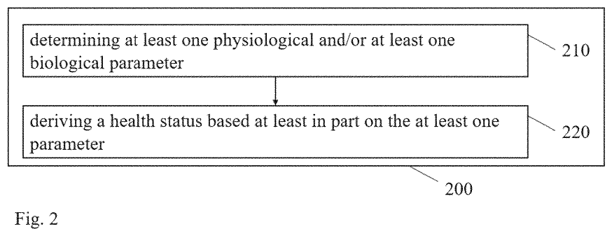

FIG. 2 Schematic representation of an exemplary embodiment of a method according to the present invention.

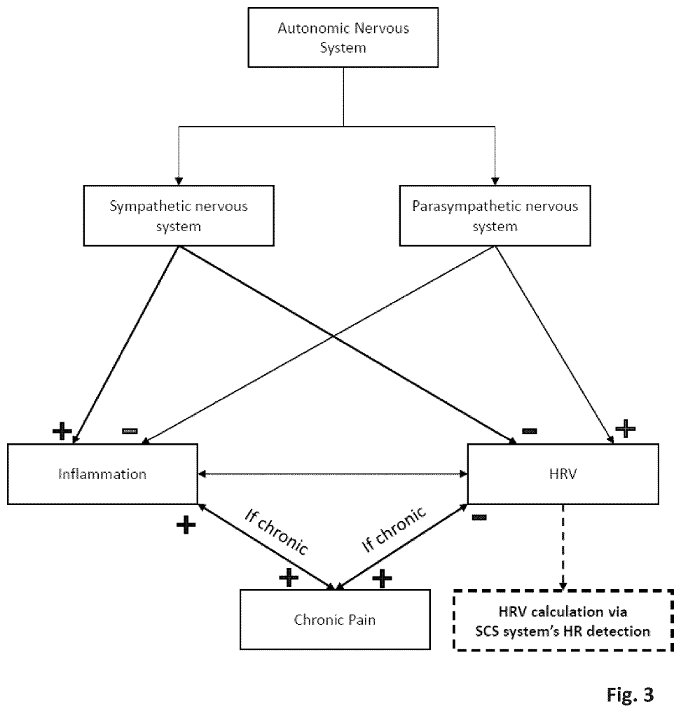

FIG. 3 Schematic representation of an exemplary embodiment of a computational method to detect changes in health status initiated by the nervous system according to the present invention.

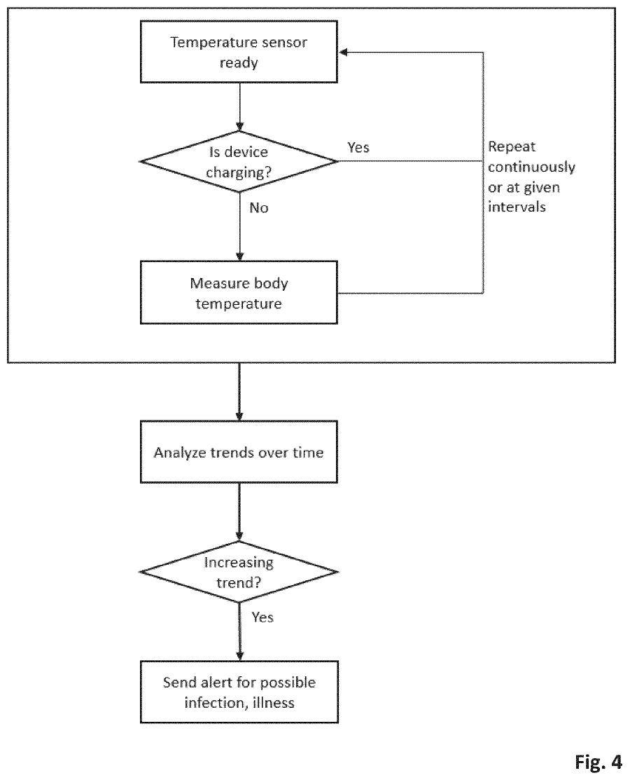

FIG. 4 Schematic representation of an exemplary embodiment of an algorithm to measure health trends and send alerts according to the present invention.

FIG. 1 shows a schematic of an exemplary neurostimulation device 100 according to the present invention.

The neurostimulation device 100 may be implanted into a patient for applying neurostimulation therapy. The neurostimulation device 100 may be configured to determine at least one physiological and/or biological parameter and derive a health status based thereon. In an example, the neurostimulation device 100 may be a spinal cord stimulator coupled to the spinal cord of the patient.

The neurostimulation device 100 may comprise a main unit which may have a power supply (e.g. a battery), as well as hardware (e.g. a control unit, power-electronics-circuitry, a storage medium, a microcontroller, a microprocessor, an analog-to-digital converter, a signal processing unit, a transceiver unit, etc.). The hardware may enable the neurostimulation device 100 to perform various computing processing steps (e.g. signal processing, various mathematical calculations, statistics, detecting signatures, etc.) which may be implemented by software (i.e. one or more computer programs), for example. The neurostimulation device 100 may have one or more leads extending from the main unit to the area to be stimulated, e.g. the spinal cord area. The leads may comprise an electrode system with one or more electrodes wherein the electrodes may be coupled to the spinal cord. The electrical pulses may be applied to the spinal cord via the electrodes to facilitate the neurostimulation to the nervous system. The neurostimulation output may be controlled by the main unit (e.g. by a control unit of the main unit) which may be coupled to the electrode system.

The neurostimulation device 100 may comprise a system S according to the present invention. The system S of the neurostimulation device 100 may comprise a temperature sensor 110 for sensing the body temperature of the patient. The temperature sensor 110 may be inside the main unit of the neurostimulation device 100. In another example the temperature sensor 110 may be situated on the outer casing of the neurostimulation device to enable an optimal coupling of the temperature sensor 110 to the surrounding body environment for an optimum pickup of the body temperature of the patient.

The system S may further comprise an evoked compound action potential (eCAP) sensor 120 for sensing the evoked compound action potential of neurons. For example, the eCAP sensor 120 may be coupled to the spinal cord to sense a variety of respective eCAP characteristics (e.g. amplitude, duration, frequency component, etc.) of spinal cord neurons. The eCAP sensor may be implemented by or included in the electrode system (comprising one or more electrodes) of the neurostimulation device which may be coupled to a particular group of nerve cells (e.g. the spinal cord). In another example, the eCAP sensor may be a separate element which comprises one or more leads extending from the main unit of the neurostimulation device 100 to the measuring area (e.g. the spinal cord). The eCAP sensor leads may comprise eCAP electrodes coupled to the nerve cells of the measuring area for picking up the respective eCAP signal.

The system S may further comprise an impedance sensor 130, e.g. a tissue impedance sensor. Alternatively or additionally to the impedance sensor, the system S may comprise a resistance sensor, capacitance sensor and/or inductance sensor. As one of these examples, the system S is further described with the impedance sensor 130. The impedance sensor 130 may be configured to determine the impedance of a tissue segment inside the patient's body. The impedance may be understood as the electrical resistance of the tissue segment for a specific electrical voltage and/or current. The tissue impedance sensor 130 may be implemented by or share elements with the electrode system of the neurostimulation device. Notably, the impedance measurement may require at least two electrodes (i.e. a pair of electrodes) for determining a respective resistance/impedance wherein the space/distance between the electrodes may define the measured tissue segment. For example, the impedance sensor 130 may extend between (pairs of) electrodes on one lead, (pairs of) electrodes between several leads, and/or between one (or several) electrode(s) and the main unit of the neurostimulation device 100 (also referred to as an implanted pulse generator), e.g. without requiring leads and/or electrodes in addition to those present for neurostimulation. This may cover a variety of tissue segments (e.g. various tissue segment lengths, various body tissues) wherein the electrode system provides a convenient readout of the impedance at a certain voltage/current between the electrodes by the neurostimulation device 100. In some examples, the tissue impedance sensor 130 may also be a separate element not sharing parts with the electrode system which is used to apply the neurostimulation therapy. For example, the tissue impedance sensor 130 may comprise one or more leads extending from the main unit to the measurement area. The leads may comprise one or more electrodes for determining a tissue impedance between at least two electrodes. This approach may give a higher degree of freedom since it is not limited to the electrode/lead system of the neurostimulation device 100 itself. For example, the leads of the tissue impedance sensor 130 may be placed in body parts not covered by the leads used for neurostimulation therapy which enhances the potentially measurable tissue segments significantly. In another example the tissue impedance sensor 130 may comprise one or more additional electrodes but also use one or more electrodes used for neurostimulation.

Further, the system S may comprise a cardiac sensor 140. The cardiac sensor 140 may be a far-field sensor for detecting a cardiac activity (i.e. heart activity) of the patient having the implant. It may be configured to sense the heart rate (e.g. HR) and/or the heart rate variability (e.g. HRV).

The system S may further comprise an activity sensor 150. The activity sensor 150 could be implemented by or comprise an inertial sensor, an accelerometer and/or a piezo sensor. It may be configured to sense any motion signal which relates to the activity metrics of the patient for subsequent signal processing and analysis.

The system S may further comprise a manual input interface 160. The manual input interface 160 may be configured for processing and/or receiving the manual entry of the at least one physiological and/or biological parameter by the patient. The manual input interface 160 may be implemented by an interface communicatively coupled to an external input device, e.g. via Bluetooth, WiFi, NFC or any other suitable wireless communication technology. It may also be conceivable to use a wired connection in some examples. For example, the external input device may be a handheld device, a personal computer, a smartphone, a smartwatch, etc.

FIG. 2 shows a schematic of an exemplary method 200 according the present invention. The method may be performed by the neurostimulation device 100 implanted into the patient. For example, the method may be implemented by a computer program running on the neurostimulation device 100 which is executing a respective algorithm.

As a starting point, the method 200 may comprise determining 210 at least one physiological and/or biological parameter. The determining 210 may be based on a sensed signal and/or a manual input.

Subsequently, the method may comprise deriving 220 a health status based at least in part on the at least one parameter.

In the following, various examples of the method are outlined regarding various parameters that may be determined and used for deriving the health status.

In an example, the health status may be derived based on a signal of a temperature sensor 110 as outlined in FIG. 1. The at least one parameter may be the body temperature of the patient which may be continuously measured by the temperature sensor 110 and thus determined by the neurostimulation device 100. In this example, the neurostimulation device 100 may determine if it is in a charging mode (e.g. a charging heating window). The charging mode may be defined as a time frame between the onset of charging and a certain duration of time after the charging has stopped, e.g. 1 hour. The charging in rechargeable neurostimulation devices 100 may be accompanied by a measurable increase of temperature in the vicinity of the neurostimulation device 100, where the temperature sensor 110 (of FIG. 1) may be located. Since the external temperature increase evoked by the charging process is not an intrinsic change of body temperature associated with the health of the patient, the body temperature may be assessed when said external influence no longer prevails. When the device determines that it is not in a charging mode, a temperature threshold may be set. The measured temperature may be continuously evaluated against the temperature threshold. If the temperature exceeds the temperature threshold for a certain duration of time (e.g. 6 hours) a health status (e.g. an indication of fever) may be determined, for example. Additionally, if the temperature threshold is crossed for the certain duration of time, a notification and/or and alert to an external device may be sent to inform medical personnel and/or the patient of the suspicious body temperature change.

In an example, the health status may be based on a signal of an eCAP sensor 120 as outlined in FIG. 1. The at least one parameter may be an eCAP feature (e.g. eCAP amplitude, eCAP duration, eCAP frequency component). The method 200 may comprise defining a set of thresholds for a set of eCAP features, wherein a set is understood to comprise at least one element, preferably at least two elements. The set of thresholds may be based on pre-operative fixed values and/or values from recordings within a patient in controlled conditions (e.g. in a state considered normal by the patient). The eCAP features may be regularly determined wherein a readout of the eCAP features may comprise a number of recordings (e.g. 100) in a recording period (e.g. 1 second). A recording may be considered a readout of raw data of the eCAP signal for further processing. Subsequently, a statistical calculation may be applied to the raw eCAP data (e.g. for determining an average value, mean value, a standard deviation), followed by calculating the eCAP features (e.g. according to the pre-defined set of eCAP features). Then, the calculated set of eCAP features may be compared and evaluated against the set of defined thresholds. The evaluation may comprise determining the number of thresholds crossed depending on the set of defined thresholds, e.g. as outlined at other passages herein. This type of evaluation may be executed by the neurostimulation device 100 once every hour. In this example, the derived health status may be based on the assessment of the set of eCAP features in an evaluation window. For example, if the evaluation of the set of eCAP features occurs once every hour and is repeated 24 times an evaluation window of 24 hours may be defined. If a certain number of evaluations indicate that a certain proportion (e.g. 70%) of thresholds were crossed over the course of the evaluation window, a corresponding health status may be derived. Subsequently, a notification and/or an alert may be sent by the neurostimulation device 100 to an external device to inform medical personnel and/or the patient of the suspicious eCAP feature characteristic.

In an example, the health status may be based on a signal of an impedance sensor 130, e.g. a tissue impedance sensor, as outlined in FIG. 1. The at least one parameter may be an impedance/resistance of a tissue segment which may be defined by the impedance sensor 130. For example, the impedance may be measured at least once daily. The method 200 may comprise considering a post-implant adaptive period wherein the impedance may not be determined for a certain time (e.g. 6 weeks) after the implantation surgery of the neurostimulation device 100 and/or the tissue impedance sensor 130. This may allow for post-operative tissue reaction to fade which minimizes the effect of acute changes of impedance due to the tissue healing. The impedance may be determined for noticeable, sustained changes. For example, the method 200 may comprise determining a sudden significant change from one tissue impedance measurement to the subsequent tissue impedance measurement (e.g. from one day to the next day). It may also comprise determining gradual changes of the impedance (e.g. over the course of several days). For this and/or all other embodiments herein impedance values over time may feed into/update a computational model using an artificial intelligence method (e.g. machine learning model), which may in turn predict current, short or long term future health status changes before changes are seen by traditional method (e.g. gradual or abrupt signal change). Based thereon, a corresponding health state may be derived. To illustrate an example, if the impedance change compared to the baseline impedance is sustained over at least two days, a notification and/or an alert may be sent by the neurostimulation device 100 to an external device to inform medical personnel and/or the patient of the suspicious impedance change.

In an example, the health status may be derived based on a manual input received by manual input interface 160 as outlined in FIG. 1. The at least one parameter may be the perception threshold (PT) or a plurality of perception thresholds (PTs) of the patient regarding one or more neurostimulation parameters. Initially, the perception thresholds may be determined with regard to the implantation surgery. The perception thresholds may be determined at time of implant and after a post-operative time period which allows the tissue reaction to fade (e.g. 6 weeks after surgery). This ensures that the subject-specific baseline of perception thresholds may be determined after surgery which may be used as a benchmark. Subsequently, the method 200 may comprise regularly determining the perception thresholds via standard clinical practices (e.g. once every week, every month, every six months, etc.). The perception thresholds may be entered manually by the patient (and/or medical personnel) and received by the manual input interface 160 as outlined with reference to FIG. 1. The entered perception thresholds may be compared to the initially determined subject-specific baseline. If the perception thresholds have changed significantly compared to the subject-specific baseline, a notification and/or an alert may be sent by the neurostimulation device 100 to an external device to inform medical personnel and/or the patient of the suspicious change of the perception thresholds.

It is noted that for sending the notification and/or alert, the same communications path may be used as that used by manual input interface 160.

In an example, the health status may be derived based on a signal of a cardiac sensor 150 as outlined with reference to FIG. 1. The at least one parameter may be a heart rate (HR) and/or a heart rate variability (HRV) which may be determined by the cardiac sensor 150 and/or the neurostimulation device 100. For example, the heart rate may be sensed and determined (e.g. recorded) at regular intervals (e.g. the HR may be recorded every hour for 1 minute). The respective heart rate variability may be calculated after each HR determination (e.g. by the neurostimulation device 100). A baseline heart rate and/or a baseline heart rate variability may be determined by evaluating these parameters over a certain time duration (e.g. 7 days, 1 month, etc.). The baseline may be updated at regular time intervals and/or continuously updated with further recordings. If a new recording of the heart rate and/or heart rate variability starts significantly deviating from the determined baseline in a sustained way (e.g. for at least 12 hours) a notification and/or an alert may be sent by the neurostimulation device 100 to an external device to inform medical personnel and/or the patient of the suspicious cardiac activity.

In another example, the HR/HRV deviation may be initially correlated with at least one other physiological and/or biological parameter as outlined herein (e.g. the body temperature, the tissue impedance, perception thresholds, etc.). The neurostimulation device 100 may be configured to calculate said one or more correlations with the hardware/software means as outlined herein. For example, a health state may be derived from one or more of such correlations and/or a corresponding notification and/or alert may be sent by the neurostimulation device 100 to the external device. For example, if the HR increase is correlated with increased activity (cf. further below), this may be considered as a normal variation. However, if the HR increase is not correlated with increased activity, or even correlated with reduced activity, a corresponding suspicious heart activity may be diagnosed, and a corresponding health state determined. This ensures that a high likelihood of a significant change in the health status of the patient may be present. Further, all above mentioned diagnosed activities and/or determined states may be fed into a machine learning algorithm that predicts health status changes (current or future).

In an example, the health status may be derived based on a signal of an activity sensor 150 as outlined with reference to FIG. 1. The at least one parameter may be based on any movement characteristic and/or activity metrics of the patient (e.g. movement type, movement type duration etc.) which may be determined based on the activity signals (e.g. activity metrics) of the activity sensor 150. The neurostimulation device 100 may be configured to continuously capture the signal of the activity sensor 150 and apply signal processing to remove signal noise. This ensures the derived/calculated parameters based from the signal have a reliable basis which enables an improved determination of the health status of the patient. The activity signal may be used for determining a variety of parameters enabling an extensive assessment of the health status wherein notifications/alerts may be sent to an external device if necessary.

In one example, the assessed health status may be based on a step count. The activity signal of the activity sensor 150 may be analyzed to identify and count steps wherein a daily step count may be recorded and monitored over time (e.g. over a month, a year, etc.). The daily step count may be stored in a database (e.g. comprised in the neurostimulation device 100) for further access. For example, the daily step counts may be used to regularly calculate statistics which may be updated with each new daily step count. The statistics may comprise an average, a standard deviation, a variance, a histogram, etc. associated with the daily step count. The neurostimulation device 100 may regularly evaluate if a sustained change and/or a significant change of the daily step count occurs which may be based on the determined statistics. When the daily step count (or a statistic associated therewith) undergoes a sustained change (e.g. for at least 4 days) and a significant change (e.g. at least a 30% difference) a notification and/or an alert may be sent by the neurostimulation device 100 to an external device to inform medical personnel and/or the patient of the suspicious change of the daily step count. Further, all above mentioned changes, notification, alerts, etc. may be fed into a machine learning algorithm that predicts health status changes (current or future).

In another example, the assessed health status may be based on a gait analysis. The activity signal of the activity sensor 150 may be evaluated by the neurostimulation device 100 to identify one or more walking bouts of the patient. Subsequently, the signal associated with the walking bout may be transformed into the frequency domain for further analysis. The walking bout frequency content may be stored in a database (e.g. comprised in the neurostimulation device 100) for further access. For example, the frequency content of the walking bout signal may be used to characterize the gait over time. This may comprise quantifying the magnitude of frequency components in pre-defined frequency windows, calculating average walking speeds and/or analyzing the walking harmonics content over a specific time frame. For monitoring the gait characteristics, the neurostimulation device 100 may regularly evaluate if a sustained deviation and/or a significant deviation of the walking bout signal (and/or walking bout frequency content) occurs. When the gait characteristics undergo a sustained deviation (e.g. for at least 3 days) and a significant deviation (e.g. at least a 30% difference) a notification and/or an alert may be sent by the neurostimulation device 100 to an external device to inform medical personnel and/or the patient of the suspicious change of the daily step count. Further, all above and later mentioned deviations, signals, notification, alerts and other information may be fed into a machine learning algorithm that predicts health status changes (current or future).

In another example, the health status may be based on a body position. The activity signal of the activity sensor 150 may be evaluated by the neurostimulation device 100 to identify common body positions (i.e. body position categories) of the patient (e.g. sitting, laying down (e.g. supine, prone, side), standing, walking, driving), while also determining the time spent in the respective body position and/or the respective acceleration signal. The associated data may be stored in a database (e.g. comprised in the neurostimulation device 100) for further access. Various statistics may be calculated based on the body position history for further analysis. For example, the proportion of time spent in each body position may be averaged over a certain period of time (e.g. a sliding window of 7 days). To illustrate an example, this evaluation may indicate that the patient spent the last 7 days 30% laying down, 40% sitting, 10% standing, 10% walking, 10% driving. A change in the time spent in a certain body position and/or in the ratios of certain body positions may be indicative of a health status. For example, the amount of walking may reduce in view of developing cardiovascular issues, etc.

In another example, the acceleration signal of a body position may be transformed into its frequency content (e.g. by a Fourier analysis) wherein the frequency content is analyzed for each position category. The frequency content of one or several body positions may be evaluated regarding a significant deviation which may be associated with a health status. Notably, the frequency content may provide information on changes in the characteristics of a body position (e.g. a different body movement (e.g. sudden accelerations through a jerk) and/or different body alignment in a body position and/or a different gait characteristic) which may correlate with the patient's health. For example, if the characteristics of a body position while sitting, standing and/or walking changes, this may be associated with a change in health status.

The change of the characteristics of a body position (i.e. the change in the frequency content) may also result in a concomitant reduced time spent in that body position, e.g., sitting, standing and/or walking, for the patient. Hence, the neurostimulation device 100 may be configured to evaluate a correlation between the proportion of time spent in a body position and the frequency content of the acceleration signal for deriving the health status.

In another example, a body-position-change-frequency may be determined which may comprise how frequent the patient switches into a body position and/or between certain body positions over a certain period of time. When the proportion of time spent in a body position, the acceleration frequency content, the body-position-change-frequency and/or a combination thereof changes significantly, a notification and/or an alert may be sent by the neurostimulation device 100 to an external device to inform medical personnel and/or the patient of the suspicious change.

In another example, the health status may be based on a sleeping pattern and/or sleeping parameter. The activity signal of the activity sensor 150 may be evaluated by the neurostimulation device 100 to identify sleeping bouts. The evaluation may comprise using a combination of current time, body position and/or body movement. For example, the evaluation may be active when the neurostimulation device 100 has determined a time associated with sleeping and/or a characteristic sleeping marker which may also be based on signals from other sensors (e.g. the activity sensor indicates a laying position, and the cardiac sensor indicates a heart rate associated with sleeping). The evaluation may further comprise identifying body movements during the night (e.g. turning and tossing, body position adjustment, rotations, sudden accelerations (e.g. jerks)) and quantifying the body movements throughout the sleep period. For example, the body movements may be quantified regarding their occurrence frequency, duration of time between movements, movement intensity and/or movement orientation, wherein further statistics may be evaluated as well. The neurostimulation device 100 may derive a critical health status based on a predetermined threshold and/or a combination of predetermined thresholds being reached by one or more sleeping parameters. For example, a threshold may be a certain number of body movements through the night with a certain intensity. The neurostimulation device 100 may further derive the critical health status based on a sustained and/or significant change of at least on sleeping parameter over a certain period of time (e.g. over a certain number of nights, e.g. over 3 nights). A combination of both aspects may also be implemented by the neurostimulation device 100 (i.e. the derived health status may be based on the predetermined threshold, as well as the sustained and/or significant change of the sleeping parameter). If the neurostimulation device 100 has derived the critical health status, a notification and/or an alert may be sent by the neurostimulation device 100 to an external device to inform medical personnel and/or the patient of the suspicious change in the sleeping pattern and/or sleeping parameter.

In another example, the deriving 220 of the health status may be based on a respiratory parameter associated with a respiratory activity of the patient (e.g. a respiratory rate). The respiratory parameter may be based on a respiratory signal sensed by a respiratory sensor comprised in the system S of the neurostimulation device 100. The respiratory parameter may also be determined based on another signal which may correlate with the respiratory activity of the patient. For example, the respiratory parameter may be determined based on the cardiac activity signal from the cardiac sensor 150.

In another example, the deriving 220 of the health status may be based on one parameter and/or any combination of the at least one parameter and/or health statuses as outlined herein. The health status may thus be based on the input of a plurality of sensors. For example, a derived health status may be based on any combination of body temperature, eCAP feature characteristic, tissue impedance, perception thresholds, cardiac activity, step count, gait characteristic, body position characteristic, sleeping pattern. The neurostimulation device 100 may be further configured to associate (e.g. correlate) the plurality of parameters with each other for the deriving of the health status. In this example, a notification and/or an alert may be sent by the neurostimulation device 100 to an external device to inform medical personnel and/or the patient of the suspicious change of a plurality of parameters, as well.

In another example, the method 200 may comprise continuously monitoring and determining/detecting when a signature and/or one or more criteria (e.g. a significant change in the at least one parameter and/or health status) are met which may correspond to a high likelihood of a sickness of the patient. This may ensure that the health of the patient is automatically tracked without requiring manual intervention by the patient.

Conclusively, the invention enables an (early) detection of the health status and/or possible illnesses in a patient population having neurostimulation implants who may be at high risk of severe health outcomes (e.g. from acute infection). Furthermore, the health status may be assessed remotely, regularly and/or automatically which contributes a valuable advantage for the patient's health beyond the primary goal of the neurostimulation therapy by the neurostimulation device 100. The early, automatic detection of physiological and/or biological cues may result in care providers reaching out proactively to patients to get an early diagnosis. Hence, it may be more likely to reduce serious short- and long-term health consequences, as well as the number of disease related deaths (e.g. virus-related deaths). The patient may thus be reliably protected from severe health consequences following an acute illness (e.g. a viral infection). The invention may improve the patient's quality of life and allow him/her to feel safer while living and/or rediscovering an independent life where caretakers cannot be closely and continuously paying attention to early signs of illness or are simply not always there to do so.

FIG. 3 shows a schematic of an exemplary embodiment of a computational method to detect changes in health status initiated by the nervous system according to the present invention. The autonomic nervous system may increase sympathetic tone in a disease state, leading to increases in tissue inflammation and decreased HRV. In contrast, recovery to a healthy state may increase parasympathetic tone leading to a reduction of inflammation and increased HRV. Chronic pain interacts with targets of the autonomic nervous, tending to further increase inflammation and decrease HRV. The present embodiment uses a sensor to measure the HR variable, computes the HRV and uses the result to make inferences regarding the chronic pain and inflammatory state of the patient.

FIG. 4 shows a schematic of an exemplary embodiment of an algorithm to measure health trends and send alerts according to the present invention. In the present embodiment a temperature sensor measures body temperature outside of device recharging intervals. Trends over time intervals are then computed from the measured temperature variable. A further algorithm analyzes the trend to detect an increase and send an alert for a possible illness and/or infection.

1. An implantable neurostimulation device (100) comprising:
a system (S) for determining one or both of at least one physiological or at least one biological parameter,
wherein the device is configured to derive a health status based at least in part on the at least one parameter,
and wherein the system (S) comprises at least one element or shares at least one element associated with a neurostimulation therapy for determining the at least one parameter.

2. An implantable neurostimulation device according to claim 1, wherein the health status comprises a likelihood of a medical condition.

3. An implantable neurostimulation device according to claim 1, wherein the device is configured to derive the health status based at least in part on one or both of a signature or a statistic of the at least one parameter over a predetermined period of time.

4. An implantable neurostimulation device (100) according to claim 1, wherein the system (S) is configured to determine the at least one parameter based at least in part on a predetermined timing.

5. An implantable neurostimulation device (100) according to claim 1, wherein the system (S) is configured to determine one or both of an abrupt change or a gradual change of the at least one parameter.

6. An implantable neurostimulation device according to claim 1, wherein the device is configured to derive the health status based at least in part on comparing the at least one parameter with a parameter specific threshold.

7. An implantable neurostimulation device according to claim 1, wherein the device is configured to monitor the health status.

8. An implantable neurostimulation device according to claim 1, wherein the device is configured to trigger an alert based at least in part on the health status.

9. Implantable neurostimulation device (100) according to claim 1, wherein the system (S) comprises at least one sensor.

10. An implantable neurostimulation device (100) according to claim 9, wherein the sensor comprises at least one of the following: a temperature sensor (110), an evoked compound action potential sensor (120), an impedance sensor (130), a cardiac sensor (140), an activity sensor (150), or an accelerometer.

11. An implantable neurostimulation device according to claim 1, wherein the at least one parameter comprises at least one of the following: a body temperature, an evoked compound action potential amplitude, an evoked compound action potential duration, an evoked compound action potential frequency component, an impedance, a heart rate, a heart rate variability, a body movement intensity, a daily step count, a gait, a body position, a sleeping pattern, or a sleeping quality.

12. Implantable neurostimulation device (100) according to claim 1, wherein the parameter comprises a perception threshold, and the system (S) is configured to determine the parameter based at least in part on a manual input to the system.

13. A method (200) carried out by an implantable neurostimulation device comprising the following steps:
determining (210) one or both of at least one physiological and/e or at least one biological parameter using a system (S), wherein the system (S) comprises at least one element or shares at least one element associated with a neurostimulation therapy for determining the at least one parameter;
deriving (220) a health status based at least in part on the at least one parameter.

14. A computer program comprising instructions to perform the method of claim 13, the instructions being configured to be executed by a computer.

15. Use of a neurostimulation device (100) implanted into a human for deriving a health status of the human, wherein the neurostimulator device (100) comprises a system for determining one or both of at least one physiological or at least one biological parameter, wherein the system (S) comprises at least one element or shares at least one element associated with a neurostimulation therapy for determining the at least one parameter, and wherein the health status is derived based at least in part on the at least one parameter.
